(19) United States
(12) Patent Application Publication
(10) Pub. No.: US 2019/0366079 A1
(43) Pub. Date: Dec. 5, 2019

(54) IN SITU ASSEMBLY OF A BI-DIRECTIONAL NEURAL INTERFACE

(71) Applicant: General Electric Company, Schenectady, NY (US)

(72) Inventors: Jeffrey Michael Ashe, Gloversville, NY (US); Craig Patrick Galligan, Niskayuna, NY (US); Christopher Michael Puleo, Niskayuna, NY (US); Tzu-Jen KAO, Niskayuna, NY (US)

(21) Appl. No.: 16/085,463

(22) PCT Filed: Mar. 13, 2017
(86) PCT No.: PCT/US2017/022041

§ 371 Date: Sep. 14, 2018

Related U.S. Application Data
(60) Provisional application No. 62/308,009, filed on Mar. 14, 2016.

(51) Int. Cl. A61N 1/05; A61B 5/04; A61N 1/36; A61B 17/00; A61L 24/10; A61L 24/08; A61L 24/06; A61L 24/04; A61L 27/18; A61L 27/16

(52) U.S. Cl. CPC A61N 1/0558; A61N 1/0556; A61B 5/04001; A61N 1/36128; A61B 17/00491; A61B 2560/0223; A61L 24/08; A61L 24/06; A61L 24/046; A61L 27/18; A61L 27/16; A61L 24/106

(57) ABSTRACT

The subject matter of the present disclosure generally relates to a method and system for providing a bi-directional neural interface having an electrode configured to be positioned on a nerve; a substrate holding the electrode; a biocompatible insulator configured to electrically isolate the electrode when the electrode is positioned on the nerve, wherein the biocompatible insulator is formed in place about the electrode; and a pulse generator configured to deliver energy pulses to the electrode.

### CROSS-REFERENCE TO RELATED APPLICATIONS

This application is a filing under U.S.C. 371 of international application number PCT/US2017/022041, filed Mar. 13, 2017, which claims the priority and benefit of U.S. Provisional Application No. 62/308,009, filed on 14 Mar. 2016, the entire disclosure of which is hereby incorporated by reference.

### BACKGROUND OF THE INVENTION

The subject matter disclosed herein relates to injectable neural stimulator/recording devices, more in situ assembly of a neural interface, e.g., a nerve cuff, and methods of stimulating and/or recording from nerves using such a nerve cuff.

Neurostimulation has been used to treat a variety of clinical conditions. For example, electrical stimulation at various locations along the spinal cord has been used to treat chronic back pain. Such treatment may be performed by a device that periodically generates electrical energy that is applied to the tissue to activate certain nerve fibers, which in turn may result in a decreased sensation of pain. In the case of spinal cord stimulation, the stimulating electrodes are generally positioned in the epidural space, although the pulse generator may be positioned somewhat remotely from the electrodes, e.g., in the abdominal or gluteal region, but connected to the electrodes via conducting wires or leads. In other implementations, deep brain stimulation may be used to stimulate particular areas of the brain to treat movement disorders, and the stimulation locations may be guided by neuroimaging. Such central nervous system stimulation is generally targeted to the local nerve or brain cell function.

Peripheral neurostimulation can be relatively more challenging than targeting the larger structures of the central nervous system. As peripheral nerves extend outwards, the size of the nerve bundle decreases. In addition, a small peripheral nerve fiber may control a comparatively large section of surrounding tissue, which makes locating and targeting such nerves for neurostimulation relatively challenging. However, the peripheral nervous system innervates many different organ structures within the body, and targeting certain peripheral nerves may be desirable.

Although implantable electrical stimulation technology has permitted co-implantation of pulse generator circuitry and integral electrodes in leadless, integrated devices implanted with control circuitry directly on the nerve, such devices present challenges to the implanted system. The integrated implant must be stably positioned on the nerve in a way that does not cause undue stress on the nerve or damage to surrounding tissue due to implant movement. However, certain implanted devices, such as nerve cuffs, that are designed to contact the nerve, cannot be positioned and used without surgical dissection of the nerve.

### BRIEF DESCRIPTION

Described herein is a nerve cuff for securing an implantable electrode device to its nerve target. The implantable electrode device may be part of a nerve cuff that is fabricated or assembled around the nerve in situ. In one implementation, the implantable electrode device (contained within a needle or surgical instrument) is first brought into contact with (or near) the nerve target using a surgical scope or external imaging technology. A biocompatible adhesive is injected around the implant at its target location to minimize relative motion (and subsequent tissue damage), and to fix the implant distance from the nerve surface. An insulating material is then injected around the adhered/fixed implant to electrically isolate the nerve-implant interface. In other embodiments, the adhesive may be applied to the desired nerve location prior to positioning the implantable electrode device. In yet another embodiment, the implantable electrode device may have a pre-applied adhesive layer such that the adhesive and electrode are positioned on the nerve at the same time. Methods of surgically implanting such nerve cuffs are also described herein, as well as methods for testing the cuff for proper placement and function.

There are numerous advantages to using an injectable or in situ formed nerve cuff, including relatively low-invasive (scope or image guided) surgical implantation and the ability to electrically isolate smaller or deeper nerves within the peripheral nervous system (i.e. those too small for surgical dissection and manipulation). The injectable materials may also serve additional functions not possible with a traditional nerve cuff. The adhesive material used to place and fix the implantable electrode device to the nerve during assembly (e.g., injection) may be also used to alter ionic conduction between the implanted electrode and nerve. This in turn may facilitate improved coupling between the electrode and the nerve and improved recording and/or stimulation. The adhesive may also be biodegradable, allowing initial protection of the nerve-electrode interface (during injection and curing of the insulation material) and then giving way to a gap or space that may be naturally filled with fluid or connective tissue (post-degradation). This material may also contain specialized components or additives (such as drugs to reduce local inflammation), and may serve to hinder high impedance tissue formation. The adhesive and insulating material may replace techniques that require suturing the nerve cuff to the nerve, a major challenge in moving from surgical dissection based implantation to low-invasive injection.

In one embodiment, the nerve cuff (once injected and assembled) includes an adhesive layer, injected between the nerve and the implanted device. This layer may be formed by utilizing a number of materials capable of in situ polymerization. These materials may also be chosen from a list currently utilized in neural surgeries, and known to have little neurotoxicity. This layer first serves as an anchor to affix the assembled nerve cuff at a fixed distance to the nerve surface. The gap spacing between the electrodes on the implant and the nerve can be adjusted during implantation through use of a “co-injected” surgical scope or external image guided system. Once affixed to the nerve, the implant can be electrically insulated by injection of a second injectable material. This material may be chosen from a list of electrical insulators capable of in situ polymerization with minimal immune response. The final assembly closely matches the functional architecture of a nerve cuff, but is assembled in situ through a needle (or surgical tool).

In one embodiment, a method of assembling a neural interface, e.g., a nerve cuff is provided that includes positioning an electrode proximate to a nerve such that the electrode is positioned partially proximate to or about an outer circumference of the nerve; applying an injectable biocompatible adhesive between the electrode and the nerve; and applying an injectable biocompatible insulator around the biocompatible adhesive, the nerve, and the electrode to provide in situ assembly of a neural interface around the nerve.

In another embodiment, a method of assembling a neural interface is provided that includes positioning an implantable electrode device comprising an electrode proximate to a nerve such that the electrode is positioned partially proximate to or about an outer circumference of the nerve and wherein the implantable electrode device comprises a biocompatible adhesive layer on a surface of the electrode such that the biocompatible adhesive layer is positioned between the electrode and the nerve after the positioning; and applying an injectable biocompatible insulator around the biocompatible adhesive layer, the nerve, and the electrode to provide in situ assembly of a neural interface around the nerve.

In another embodiment, a kit for positioning an electrode on a nerve is provided. The kit includes a first applicator chamber holding a biocompatible adhesive a second applicator chamber holding a liquid or gel material that forms a biocompatible insulator; and an implantable electrode device positioned proximate to the first tip and the second tip. The kit may be used in conjunction with a surgical scope or external image-guided system for low-invasive implantation of the neural implant through a small incision.

In another embodiment, the implant is positioned without the use of the first adhesive material, and the implant itself protects the gap between the implant electrodes and nerve during injection of the insulator material. In this embodiment, the insulating material must also provide support to affix the implant to the nerve with little relative motion, and must be compatible with direct nerve contact.

In another embodiment, the implant is positioned without the use of the second insulating material, and the implant itself (or an injectable support structure) provides a means of directing the electrical current to the nerve. In this embodiment, the adhesive material must provide the support to affix the implant to the nerve with little relative motion, and must be compatible with direct nerve contact.

Finally, in one embodiment a system for a bi-directional neural interface is provided. The system includes an electrode configured to be positioned on a nerve; a substrate holding the electrode; a biocompatible insulator configured to electrically isolate the electrode when the electrode is positioned on the nerve, wherein the biocompatible insulator is cured in place about the electrode; and one or more of a pulse generator configured to deliver energy pulses to the electrode or a recording device. For example, a separate sensing circuit may be contained within the system that may utilize electrodes as ground and sensing electrodes to record electrical signals corresponding to natural or stimulated neural activity.

### BRIEF DESCRIPTION OF THE DRAWINGS

These and other features, aspects, and advantages of the present disclosure will become better understood when the following detailed description is read with reference to the accompanying drawings in which like characters represent like parts throughout the drawings, wherein:

FIG. 1 is a detailed perspective view depicting a nerve cuff including an implantable electrode device positioned on a nerve after injection and in situ assembly, according to embodiments of the disclosure;

FIG. 1B is a cross-section of the injected nerve cuff of FIG. 1A;

FIG. 1C is a cross-section of the injected nerve cuff according to an alternative embodiment of the disclosure;

FIG. 2A is a front-of an assembled nerve cuff including the implantable electrode device positioned on a nerve after injection and in situ assembly, according to embodiments of the disclosure;

FIG. 2B is an embodiment of the cuff design using a non-degradable adhesive;

FIG. 2C illustrates an embodiment of the cuff design using a degradable adhesive;

FIG. 3A is a side perspective view of an assembled nerve cuff;

FIG. 3B is a side view of the assembled nerve cuff;

FIG. 3C is a longitudinal section through the assembled nerve cuff attached to or in position on a nerve, showing internal features s;

FIG. 4A is a schematic of an example peripheral nerve location and two surgical incisions used to gain access to the nerve for injection of nerve cuff and placement of a surgical scope,

FIG. 4B is a view of the injection site after insertion of the implant injection device and surgical scope,

FIG. 4C is a view of the surgical scope providing an image of the local target nerve location for location of the implant injection device;

FIG. 4D illustrates actuation of the liquid nerve adhesive material prior to placement on the target nerve;

FIG. 4E illustrates the implant injected and fixed on the nerve after polymerization and curing of the nerve adhesive material;

FIG. 4F illustrates the second injection of the electrical insulation material around the implant/nerve/neural adhesive construct;

FIG. 4G illustrates completion of the assembly of the nerve cuff after polymerization and curing of the electrical insulation material;

FIG. 4H illustrates completion of the injection process by removal of the implant injection device and scope and suturing of the incisions;

FIG. 5A illustrates an embodiment of the nerve cuff in which the portion contacting the nerve is shaped like a letter J to increase the surface area of contact;

FIG. 5B illustrates the nerve cuff of FIG. 5A in position on the nerve;

FIG. 5C illustrates an embodiment of the nerve cuff in which the portion contacting the nerve is a partial annulus to increase the surface area of contact;

FIG. 5D illustrates the nerve cuff of FIG. 5C in position on the nerve;

FIG. 6A illustrates an embodiment of the injection device used for in situ fabrication of the nerve cuff in which the channels for material injection have a geometry specified for serial application of two material components;

FIG. 6B illustrates an embodiment of the injection device used for in situ fabrication of the nerve cuff in which the materials are pre-loaded sequentially in the injection device for serial application of two material components;

FIG. 6C illustrates an embodiment of the injection device used for in situ fabrication of the nerve cuff in which the nerve adhesive is pre-loaded around the implant and the electrical insulation material is loaded sequentially within the device;

FIG. 7A illustrates an embodiment of an assembled nerve cuff with a bi-polar electrode arrangement;

FIG. 7B illustrates an embodiment of the assembled nerve cuff with a tri-polar electrode arrangement;

FIG. 7C illustrates an embodiment of the assembled nerve cuff with an alternative electrode arrangement;

FIG. 7D illustrates an embodiment of the assembled nerve cuff with an electrode array;

FIG. 7E illustrates an embodiment of the assembled nerve cuff with a single electrode in contract with the nerve, where a second ground electrode is placed at a distance in surrounding tissue;

FIG. 7F illustrates an embodiment of the assembled nerve cuff with a wireless electrode configuration capable of power and/or data transfer;

FIG. 8A illustrates an embodiment of an injection device with features protruding from the injection needle or instrument;

FIG. 8B illustrates the injection device of FIG. 8A during assembly of the nerve cuff;

FIG. 9 illustrates an embodiment of the injection device in which features protruding from the injection needle or instrument are removable and remain with the implant after in situ assembly of the nerve cuff;

FIG. 10 is a block diagram of a stimulating neural device within the injectable system according to embodiments of the disclosure;

FIG. 11A depicts electrode positioning on a 70 um nerve;

FIG. 11B depicts an injected and assembled nerve cuff positioned on a 70 um nerve;

FIG. 12A shows neural recording data of an action potential before, assembly of the nerve cuff components;

FIG. 12B shows neural recording data of an action potential during injection and assembly of the nerve cuff components; and

FIG. 12C shows neural recording data of an action potential after assembly of the nerve cuff components.

### DETAILED DESCRIPTION

One or more specific embodiments will be described below. In an effort to provide a concise description of these embodiments, not all features of an actual implementation are described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure

The present techniques relate to creating an injectable bi-directional neural interface or nerve cuff capable of interfacing with peripheral nerves for directed stimulation and/or neural recording without full surgical dissection of the target nerve. In particular, the disclosed bi-direction neural interface permits one or both of stimulation or recording capabilities in an injectable format by achieving anchoring and electrical insulation without the need to physically wrap a nerve in a pre-fabricated nerve cuff. The techniques further provide improved positioning of a bi-directional neural interface at small peripheral nerves that cannot be accessed by traditional nerve cuff technologies. In one embodiment, the nerve structure may be a peripheral nerve.

Certain techniques for creating bidirectional neural interfaces/implants involve surgical dissection of a nerve and physically insulating the nerve with a silicone sheath. These neural interfaces are called neural cuffs. However, neural cuffs are difficult to scale down in size to be applied to smaller nerves because the application involves physically wrapping a plastic insulating sheath around an exposed nerve. Such size limitations prevent use of cuffs in relatively smaller peripheral nerves. Simpler interfaces have been developed for unidirectional or stimulation only neural interfaces, including injectable implants that do not directly interface with a nerve (but are injected in the nerves proximity). However, these “cuffless” injectable devices do not concentrate current toward the nerve during stimulation (current is projected everywhere and not focused by an insulating cuff material), and therefore have higher current and power requirements (and may cause tissue damage). In addition, the “cuffless” devices cannot perform neural recording as the electrodes are not insulated from signals originating from surround/non-neural tissue.

Provided herein are techniques for creating an injectable bi-directional neural interface. In one embodiment, an implantable electrode device is aligned with a small injection needle and positioned on the nerve of interest. Then, a small amount of biocompatible adhesive is applied. This step attaches the electrode to the nerve at a correct location/distance and provides mechanical support without full surgical dissection of the nerve. The biocompatible adhesive may be a gel or hydrogel and maintain transport properties that allow for movement of ions and molecules necessary for maintaining health of the nerve and monitoring and/or stimulating electrical activity. Subsequently, a biocompatible insulator) is injected around the electrode/nerve interface. This can be accomplished with the same needle (sequential injection) or with two different needles in the implantable electrode device. In addition, the needles may be shaped to fit around the nerve, and the technique may provide a mold or support for shaping the injectable materials and ensuring encapsulation of the interface. The nerves may be relatively small, e.g., as small as 1 mm.

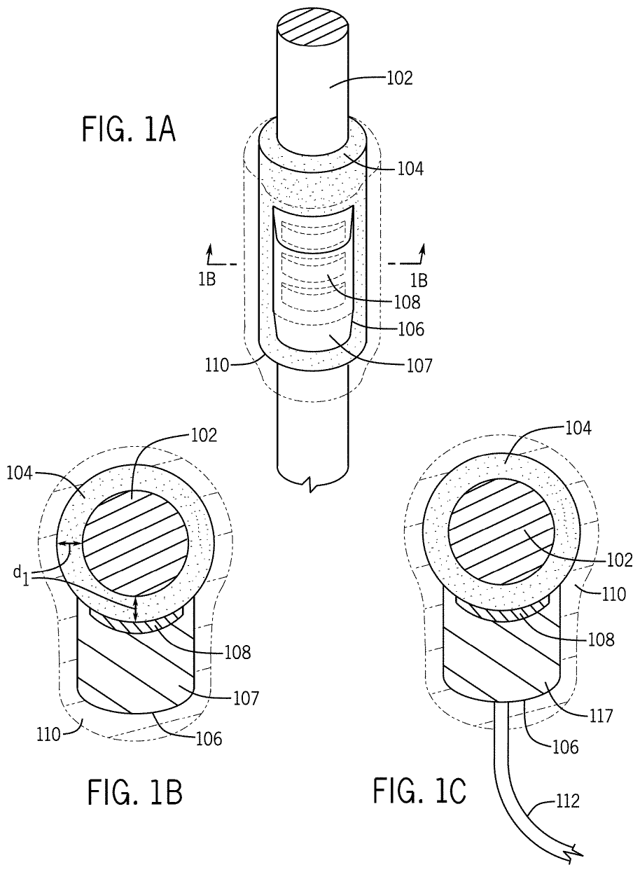

FIG. 1 is a schematic representation of an assembled nerve cuff 100 that forms a bi-directional neural interface comprising an implant positioned at a desired location on a nerve according to the disclosed techniques. The desired positioning and location of the implant positioned on the nerve may be selected to facilitate stimulation and/or recording at a desired tissue location. It should be understood that the bi-directional neural interface may include electrodes positioned at any suitable nerve location, e.g., peripheral nerves, central nerves. In one example, the nerve locations may include intracostal nerves, subcostal nerves, the brachial plexus, the lumbar plexus, the sacral plexus, the femoral nerve, the sciatic nerve, the saphenous nerve, the tibial nerve, the peroneal nerve, the ulnar nerve, the obturator nerve, the genitofemoral nerve, the median nerve, the iliohypogastric nerve, the radial nerve, the musculocutaneous nerve, etc. Further, the positioning on the nerve may be at any suitable location supported by accessibility and nerve size.

FIG. 1A is an example of a nerve cuff 100 in position following in situ assembly (e.g., injection) on or proximate to a nerve 102. In general, the nerve cuff 100 is formed in situ so that the assembled nerve cuff 100 contains an injectable insulator 110 that surrounds an implantable electrode device 106 that is anchored and in certain embodiments, protected by an injectable neural adhesive 104. The implantable electrode device 106 contains one or more integrated electrodes 108 on the surface, e.g., a substrate 107, of the device and proximate to the nerve 102. 108 or for recording nerve activity.

As illustrated in FIG. 1A, the device 106 is implanted to partially wrap around the nerve, so that electrodes 108 are positioned in close proximity to the nerve and protected by the neural adhesive 104 prior to injection and encapsulation with the insulating material 110. Because the electrodes 108 may be on or slightly separated from the nerve 102, the relatively close and stable positioning may also protect the nerve by permitting use of relatively lower power levels for stimulation, due to the fixed proximity of electrodes and current focusing provided by the insulating material 110. As provided herein, an electrode 108 or implantable electrode device 106 that is proximate to the nerve 102 may be in direct contact (complete or partial) or separated from but in close proximity to the nerve 102. For example, an electrode 108 in the assembled nerve cuff 100 may be spaced apart from the nerve 102 a distance of less than 5 mm, 3 mm, 1 mm, 100 microns, or 50 microns. In another embodiment, the adhesive 104 is in direct contact with the nerve 104 while the insulator 110 may have no or limited contact with the nerve 102. That is, as compared by total surface area of contact, the adhesive 104 has greater surface area in contact with the nerve 102 relative to the insulator 110.

In contrast to traditional sutured cuffs, the use of a biocompatible adhesive 104 permits a reduction in the distance d₁ between the electrode 108 and the nerve 102. Typically, in a traditional cuff, the space between the cuff and the nerve fills with interstitial fluid, e.g., to fill a gap of about 100 microns. However, in the disclosed embodiments, the presence of the adhesive materials blocks or reduces migration of the interstitial fluid into the space between the electrode 108 and the nerve 102 and allows a decrease in the gap d₁ between the nerve 102 and the electrode 108. This decrease in the gap relative to other techniques permits improved recording and/or stimulation functionality. With regard to recording, a recording electrode may record electrical activity from the nerve 102 plus undesired electrical activity from muscle/tissue (greater than nerve). The biocompatible adhesive 104 may be relatively breathable, with a pore size that permits ion conduction to conduct electrical activity along the nerve 102, while the polymer material itself may be relatively insulating. In contrast, the biocompatible insulator 110, when cured, has a smaller pore size than the biocompatible adhesive 104. Selection of particular biocompatible adhesive 104 and/or biocompatible insulator 110 allows for customization of the electrical environment around the nerve 102. In one embodiment, the biocompatible adhesive 104 is capable of more water absorption than the biocompatible insulator 110. In another embodiment, the biocompatible insulator 110 is relatively more hydrophobic than the biocompatible adhesive 104.

Referring to FIG. 1B, the insulator 110 surrounds a space that includes the implantable electrode device 106 and the nerve 102. In one embodiment, the insulator 110 encapsulates the implantable electrode device 106 on the nerve 102. Prior to encapsulation, the device 106 is anchored to the nerve using an injectable adhesive 104. This adhesive also protects any intervening space between the nerve 102 and electrodes 108 by filling in the gap and thus prohibiting the intervening space from being filled with the electrically insulating material of the insulator 110 as the insulator 110 is applied. This is turn enhances electrode-nerve contact by preventing insulation between these elements (e.g., to permit energy pulses from the electrode 108 to be applied to the nerve 102 and/or to permit recording of nerve electrical activity by the electrode 108) while also permitting insulation of the electrode 108 from the electrical activity of the surrounding tissues. In one embodiment, the implantable electrode device 106 is wirelessly powered and contains a microcontroller that serves as a pulse generator to control the number, timing, and type of electrical pulses applied to the nerve 102. In another embodiment, the wireless element of the implantable electrode device 106 is also utilized to communicate data. In this case the implantable electrode device 106 may also contain the circuitry necessary to record energy (or neural signals) produced by the nerve. FIG. 1B shows an alternative embodiment in which an insulated electrical lead 112 may protrude from the nerve cuff 100 and replace wireless power and communication.

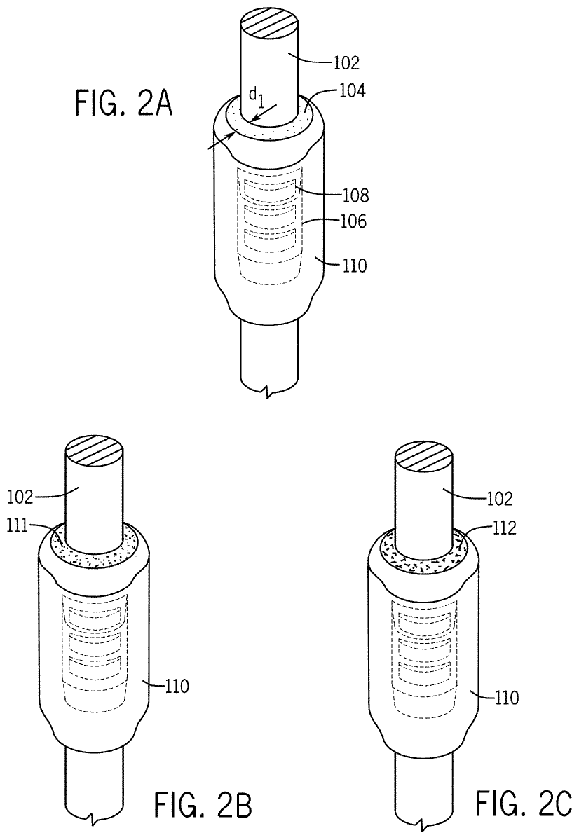

FIG. 2A illustrates the surface of the injected nerve cuff 100 after in situ assembly. Once assembled, the insulator 110 is exposed and forms the exterior layer of the nerve cuff 100. The implantable electrode device 106 is fully encapsulated and shielded from the surrounding tissue except at the gap d₁ between the electrode 108 and the nerve. In certain embodiments, the gap d₁ may be about the same as a gap between an edge of an opening 109 of the insulator 110 and the nerve 102 that is filled by the adhesive 104. That is, the gap between the nerve 102 and the electrode 108 or the insulator 110 may be of a similar size. The gap between the nerve 104 and the insulator 110 (or electrode 108) may vary along the length of the insulator, depending on the general form of the insulator shell, which in turn may be influenced by injection parameters. In the depicted embodiment, the insulator 110 forms a cylindrical layer with one or more openings 109 that permit entry of interstitial fluid into the partially enclosed space formed by the insulator 110. The interstitial fluid may flow into pores formed in the adhesive 104 or may generally be absorbed by the adhesive 104. The entry of fluid into the partially enclosed space facilitates nerve health and also may enhance conduction of the energy from the electrodes 108. Unlike a traditional nerve cuff (in which the gap between the electrodes and nerve is left empty), the adhesive layer may be chosen to provide a specific porosity and ionic conductivity. For neural recording, these parameters (i.e. transverse conductivity along the cuff) may be tailored to decrease noise signals associated with ionic/electrical activity of surrounding tissues. The adhesive layer may also be used to release steroids or other molecules to control local inflammatory response and decrease the formation of high impedance tissue between the electrodes and nerve. Electrical and impedance sensing circuits within the implant may be used to monitor the fit of the injectable cuff during injection/implantation or during subsequent periods of adhesive degradation or tissue infiltration.

The gel or other biocompatible adhesive 104 may facilitate correct placement of the implant device 106 on the nerve 102 and maintain appropriate contact during natural movement of the nerve and/or adjacent tissue. For example, nerves may move within the body as a result of circulatory volume changes or tissue movement. The disclosed adhesion and insulation technique may, in certain embodiments, permit micromovement of the implant device 106 and the nerve without affecting the integrity of electrode placement. While traditional techniques employ suturing of an electrode in place to reduce the chance of migration away from the desired site, the disclosed techniques achieve targeted electrode placement without the additional procedural steps of suturing, which remain difficult to perform on small internal nerves.

As provided herein in the disclosed embodiments, the biocompatible adhesive 104 may be a liquid, foam, or gel capable of being applied via injection (i.e., injectable) or pre-applied to the electrode in a layer. In certain embodiments, the biocompatible adhesive may be cured, polymerized, activated, or formed in situ. The material of the biocompatible adhesive 104 may be selected to have a desired viscosity or flow properties to be capable of being applied with a desired force (e.g., operator or mechanical force through an application tip or needle). In one embodiment, the viscosity, measured at 25° C., of the biocompatible adhesive 104 is between 80-60,000 cP. In certain embodiments, the biocompatible adhesive 104 includes one or more of fibrin, chitosan, polyvinylpyrrolidone, pyroxylin/nitrocellulose or poly(methylacrylate-isobutene-monoisopropylmaleate), or an acrylate or siloxane polymer.

The biocompatible adhesive 104 may include certain additional preservative or modifying components, such as a rheology modifying agent in the form of a solvent, a non-volatile diluent, and/or a volatile diluent. Examples of suitable solvents include dimethylsulfoxide (DMSO), dimethylformamide (DMF), tetrahydrofuran (THF), glyme, and combinations thereof. Examples of suitable non-volatile diluents include dimethylsulfoxide (DMSO), propylene carbonate, diglyme, polyethylene glycol diacetates, polyethylene glycol dicarbonates, dimethylisosorbide, and combinations thereof. Examples of suitable volatile diluents include hydrocarbons, perfluoroalkanes, hydrofluoroalkanes, carbon dioxide, and combinations thereof. The adhesives may also include one or more stabilizers. Examples include antioxidants (e.g., BHT and BHA), water scavengers (e.g., acyl and aryl halides, and anhydrides), Bronsted acids, and the like. In certain embodiments, the biocompatible adhesive 104 may be electrically conductive to permit the electrical signals from the electrode to be conducted to the nerve through the adhesive material. However, in embodiments in which the biocompatible adhesive degrades, the conductive properties may vary. In certain embodiments, the resistive properties of the biocompatible adhesive may result in improved readings.

The biocompatible adhesive 104 may be cured or formed in place once applied. That is, the material is applied via an applicator tip or needle and may be in a precursor form that is subsequently formed or cured by time, temperature, energy application, or the addition of an additional compound. In certain embodiments, the cure time of the biocompatible adhesive 104 may be selected to be less than five minutes to limit the exposure of the nerve 102 during assembly of the nerve cuff.

The biocompatible insulator 110 may be a silicone polymer that is applied in a precursor liquid or gel form and that hardens or cures in place such that the cured insulator 110 is harder than the adhesive 104 when both are in final form for use in the nerve cuff 100. For example, the biocompatible insulator 110 may be Master Bond Mastersil 151 encapsulant or Master Bond EP30DP or similar polymer encapsulate, available from Master Bond (Hackensack, N.J.). The biocompatible insulator 110 or its precursor material may have similar flow properties to the biocompatible adhesive and may be capable of being injected (i.e., injectable), with a viscosity that permits application via the applicator tip and flow around the electrode 108 to form a biocompatible insulator shell that encapsulates the electrode 108.

FIG. 2B depicts the injectable neural cuff after implantation using a stable or non-degradable adhesive 111 which remains intact throughout implant lifetime. During implantation, the adhesive 111 flows around the implantable electrode device 106 and may separate the implantable electrode device 106 from direct contact with. The non-degradable adhesive 111 may limit movement of the nerve cuff up and down the nerve, and eliminate friction due to movement between the device and nerve. The adhesive material (e.g., adhesive 104 or non-degradable adhesive 111) and surrounding insulator 110 may be chosen from material that has a low stiffness and absorbs shock produced by movement of surrounding tissues, while still eliminating extreme strain of torsion of the encapsulated nerve. FIG. 2C shows an alternative embodiment in which the adhesive 104 is degradable, and upon degradation leaves the device 106 and electrodes 108 suspended within the space formed by the insulator 110. In this case, the gap d₁ may be filled with fluid and/or connective tissue.

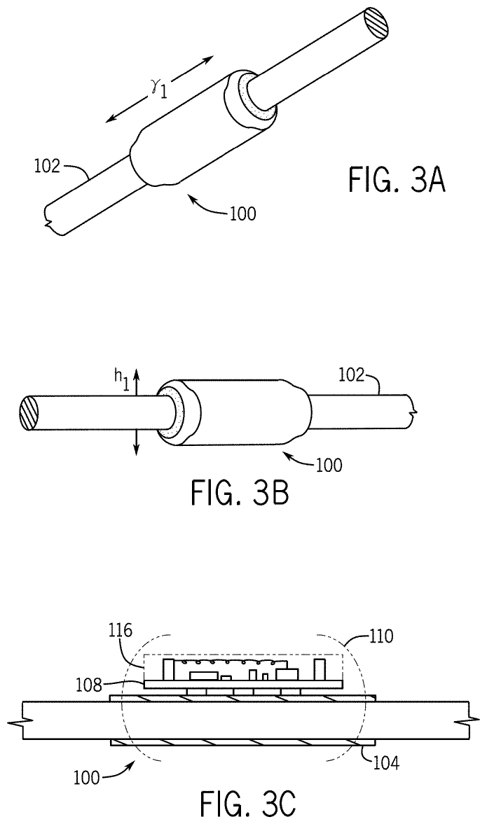

FIGS. 3A and 3B shows a perspective view of the assembled nerve cuff 100 with exemplary dimensions h₁ and l₁. In the depicted embodiments, the length l₁ along the nerve 102 may be longer than the height dimension h₁. The dimensions l₁ and h₁ of the assembled nerve cuff 100 depend on the volume of applied adhesive and insulator and may vary depending on patient physiology (e.g., the nerve environment) and operator application techniques. The suture-free nerve cuff 100 may be utilized to implant neural recording and stimulation devices on small/internal nerves (e.g., via miniaturized devices). FIG. 3C depicts an example cross-sectional schematic of an assembled nerve cuff 100 containing on-board electronics 116, such as a microcontroller, antenna, and power storage and stimulation circuit.

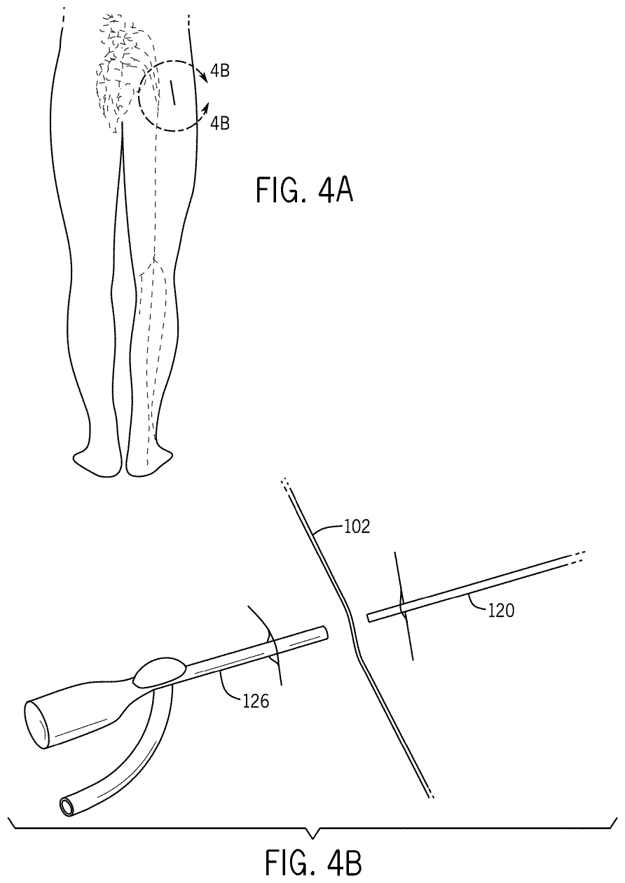

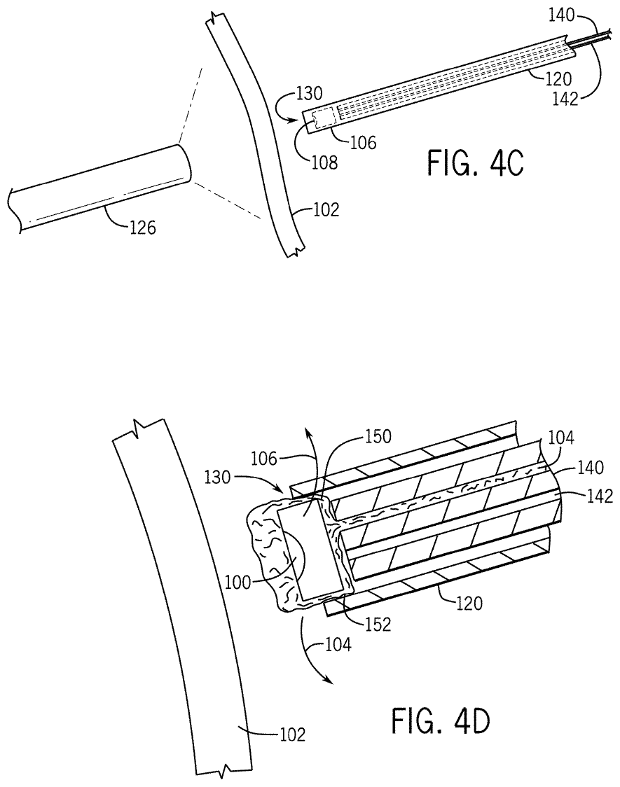

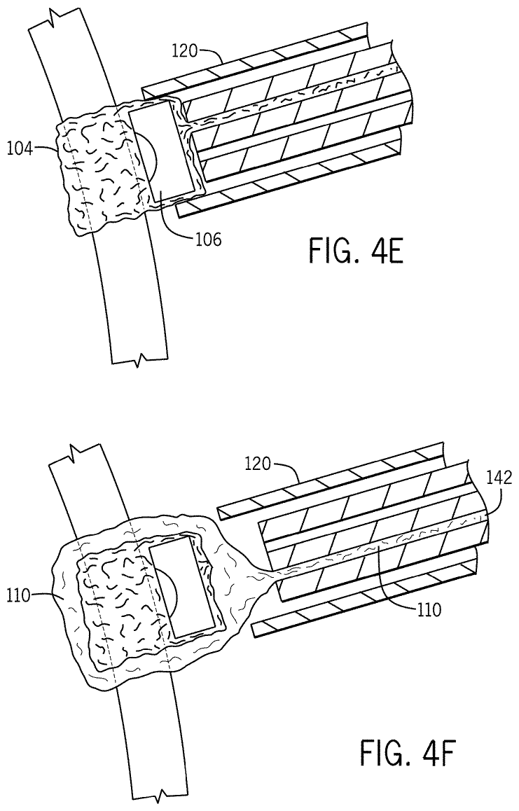

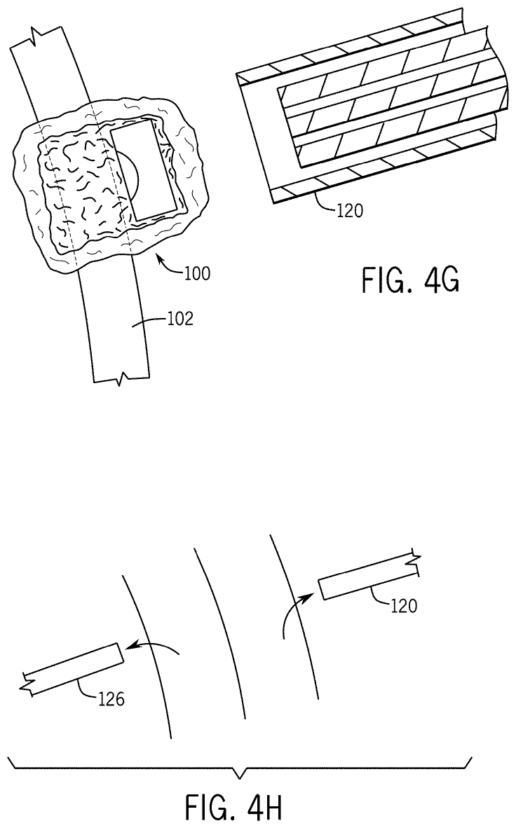

FIG. 4A is an illustration of the first step of the injection process for in situ fabrication of the nerve cuff. The illustration uses the sciatic nerve as an example nerve target. Two incisions are made on either side of the target nerve, one for insertion of the surgical instrument or injection device 120 used for injecting the nerve cuff/implant and another for insertion of a surgical scope 126 used to visualize implant placement and nerve cuff assembly. FIG. 4B illustrates insertion of the surgical tool and scope in reference to the target nerve. In another embodiment, insertion of the surgical instrument may be performed using image guidance and the second incision for the surgical scope may be eliminated.

To position the electrode 108, the surgical injection device 120 is brought into proximity with the patient's nerve 102 as shown in FIG. 4C. The injection device 120 may be shaped like a needle to aid in insertion toward the target nerve 102. The implantable electrode device 106 contained at the tip 130 of the injection device 120 is positioned at the desired location on the nerve 102. The injection device 120 may include an applicator (used to push and position the implant at the electrode); this positioning may be viewed through the surgical scope (or non-invasive imaging modality). The applicator may contain internal channels 140, 142 for application of the injectable adhesive and insulating materials or the materials may be injected using separate needles and syringes. The channels 140, 142 may be coupled to respective reservoirs of the injectable adhesive and insulating materials (e.g., adhesive 104 and insulator 110).

Once the electrode is in position, the biocompatible adhesive 10 is applied through the channel 140 directly to the implantable electrode device 106102 via an applicator tip 13 positioned adjacent to the nerve 102 as shown in FIG. 4D. The biocompatible adhesive 10 fills in and/or forms a gap between the electrode 108 and the nerve 102. In this manner, the biocompatible adhesive 104 is positioned between the nerve 102 and the electrode 108. As shown in FIG. 4E, the application force of the biocompatible adhesive 104 pushes the biocompatible adhesive 104 into place and around the electrode 108 to the nerve. The biocompatible adhesive 104, when in position, may substantially surround both the nerve 102 and the electrode 108, forming a nerve-electrode-adhesive complex. After the biocompatible adhesive 104 is applied, and cured if necessary, the second channel 142 is used to apply a biocompatible insulator 110 to the nerve-electrode-adhesive complex as shown in FIG. 4F. The channels 140, 142 include respective openings 150, 152 through which the injectable materials are pushed and applied. The biocompatible insulator 110 is then cured, cross-linked, or hardened in place around the electrode 108.

In operation, the channels 140, 142 may be filled with the biocompatible adhesive 104 or the biocompatible insulator 110 and applied by actuation of, for example, syringe plungers. The application may also be accomplished by robotic or mechanical control of the injection device 120. In one embodiment, the second applicator tip 68 may be positioned to align with the first application tip such that their respective exit ports are about the same distance away from the electrode. In examples in which the second applicator tip is positioned by the operator, the implantable electrode device may include alignment or guiding features to facilitate positioning of the exit port of the second applicator tip with an appropriate distance from the electrode 108.

After the nerve cuff 100 is assembled in situ, the biocompatible adhesive 104 and the surgical injection device 120 is removed as shown in FIG. 4G, leaving the assembled nerve cuff 100 on the nerve 102. The image device 126 and injection device 120 may be removed via the access sites to complete the assembly procedure.

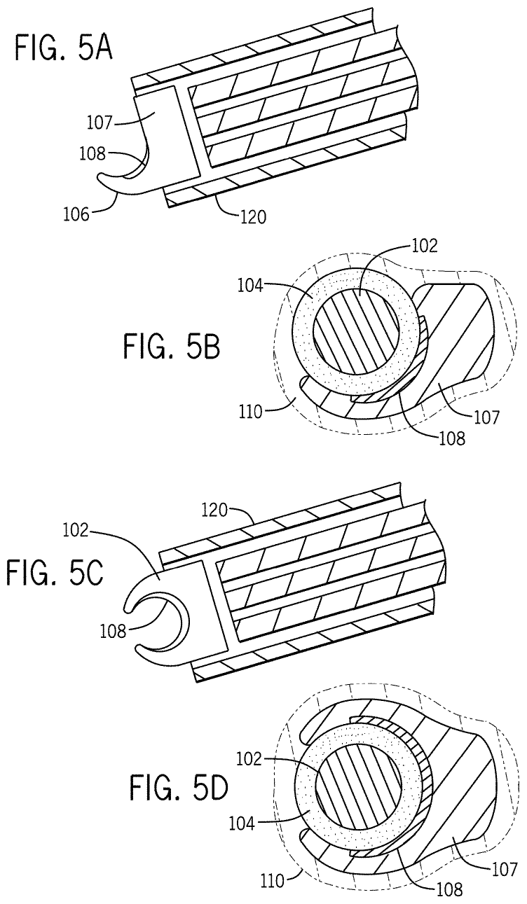

FIG. 5A shows a configuration of the implantable electrode device 106 including a curved or hook-shaped electrode 108 and substrate 107. The hook-shape may serve to capture the nerve 102. FIG. 5B is a view of the implantable electrode device in position on the nerve 102. FIG. 5C is a view of another embodiment in which the implantable electrode device 106, which forms a partial annulus about the nerve 102 when in place, as shown in FIG. 5D.

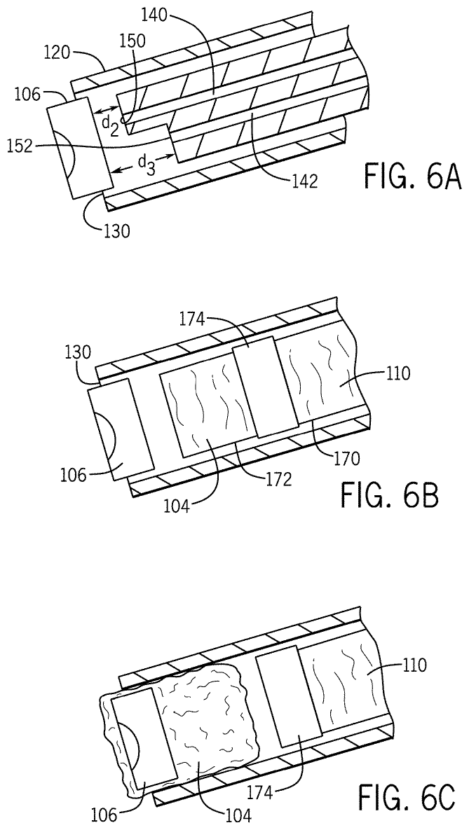

The injection device 120 may include one or more channels (e.g., channel 140, 142) through which the adhesive 104 and/or the insulator 110 are applied. The channels may be fixed or formed within the injection device 120 or may be removable. Further, the channels may be coupled to syringes holding the appropriate material for application that are actuated by an operator. In certain embodiments, for example as shown in FIG. 6A, the channel opening positions of the application channels may be positioned to facilitate serial placement of the adhesive 104 and the insulator 110. For example, the channel opening 150 associated with the adhesive channel 140 may be slightly recessed relative to the channel opening 152 from which the biocompatible insulator 110 is applied. In this manner, the adhesive 104 may be encourage to be as close as possible to the nerve during application. injection device 120 channels 140, 142 or alignment In an alternative embodiment, an injection device 120 may be coupled to two separate applicators (e.g., reservoirs) that may be positioned or used in series. The applicators may be syringes that couple to the appropriate channels of the injection device (e.g., channel 140, 142) or may be used in place of the channels. For example, a first applicator may be used, then removed, and replaced with a second applicator. In yet another embodiment, as shown in FIG. 6B, a single applicator 17 with a serial-chamber configuration may be used. For example, a first chamber 72 that is positioned closer to the applicator tip 130 holds the biocompatible adhesive while a second chamber 70 located more distally holds the biocompatible insulator 110. The chambers 170, 172 are separated by a seal 74 that may be breached to release the contents of the second chamber 70 once the first chamber 74 is empty, as shown in FIG. 6C. In this manner, both the biocompatible adhesive 104 and the biocompatible insulator 110 may be applied with a single application, which may be advantageous for placement of electrodes on smaller peripheral nerves where there may not be space for two applicators. In another embodiment, the adhesive material may be pre-applied around the implant and placed on the target nerve already encapsulated in the adhesive material.

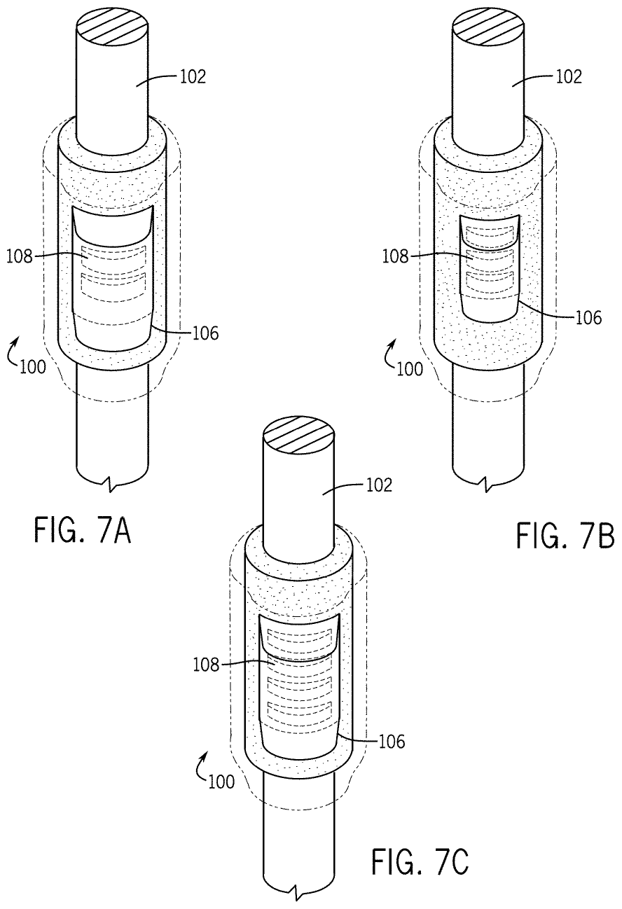

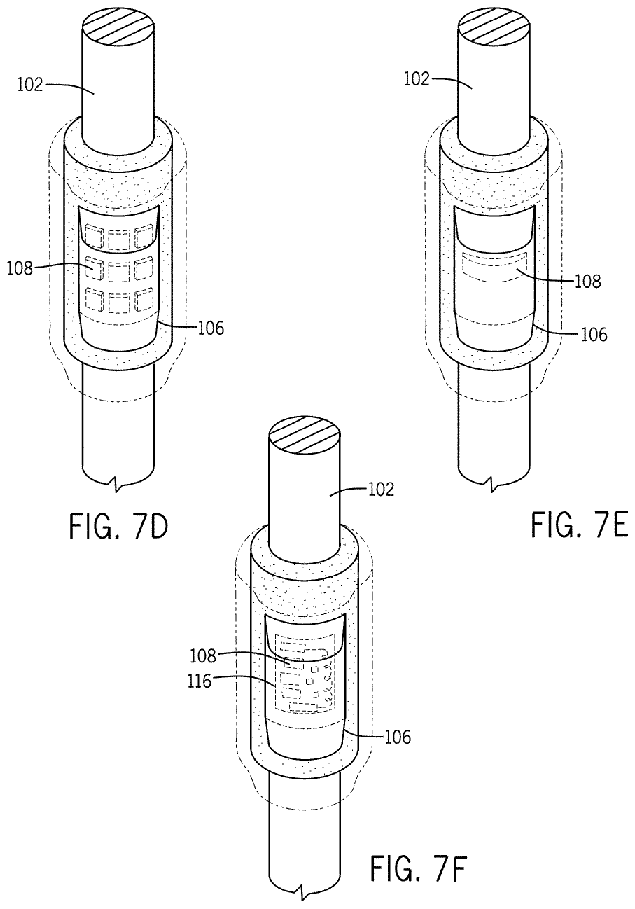

Various arrangements of electrodes 108 are contemplated according to the present techniques. Accordingly, the electrodes 108 may be positioned on the nerves in any suitable arrangement. FIG. 7A is a dual electrode configuration, FIG. 7B is a tri-electrode configuration, FIG. 7C is a four electrode configuration, and FIG. 7D is an example of the nerve cuff 100 including a micro-multielectrode array. Further, the electrodes 108 may be placed on substrates that also include electronics 116, e.g., control and power electronics and wireless communication circuitry, i.e., an antenna as in FIG. 7F. FIG. 7E is a single electrode configuration. The disclosed electrodes 108 may be in any suitable arrangement of recording, stimulating, and/or ground electrodes. In addition, the disclosed techniques may be use to separately place one or more electrode assemblies on the nerve.

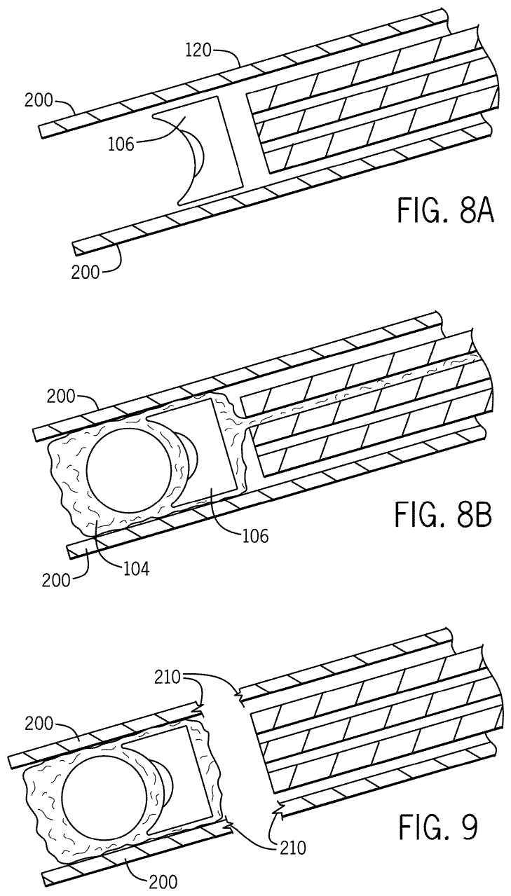

FIG. 8A shows an injection device with extending portions 200 that, when in position, may partially surround the nerve 102, as shown in FIG. 8B. The extending arms 200 act to direct the flow of the injected material, e.g. the biocompatible adhesive 104, to the area of the nerve and the implantable electrode device 106. FIG. 9 shows an alternative embodiment in which the extending arms break away from the injection device 20 (e.g., at scored break points 210) and remain with the assembled nerve cuff 100.

In one embodiment, various components used to perform the disclosed techniques may be provided to operators as a kit. The kit may be packaged and sold as a unit, and may include the appropriate injection device 120, which may be provided pre-filled with the biocompatible adhesive 104 (or its precursor material) and the biocompatible insulator 110 (or its precursor material). As noted, the biocompatible adhesive and/or the biocompatible insulator 110 may be formed in place, via exposure to cross-linking or curing agents, and such agents may also be provided as part of the kit. The kit may include the implantable electrode device 106 within or separate from the injection device 120. The kit may further include the imaging device 126.

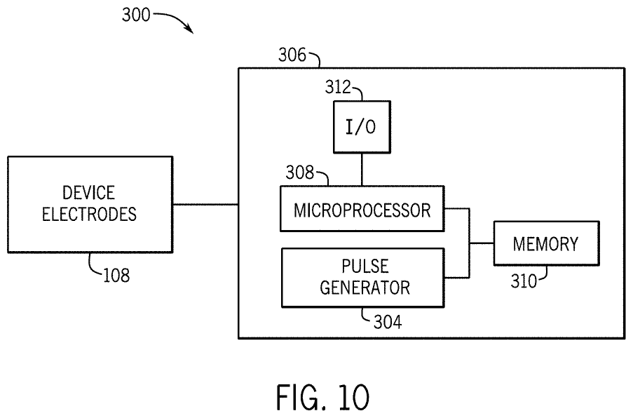

As shown in FIG. 10 a system 300 utilizing a bi-directional neural interface may include a pulse generator 304 that is adapted to generate energy pulses for application to a tissue or nerve of a patient. The pulse generator 304 may be implantable or may be integrated into an external device, such as a controller 306. The controller 306 includes a processor 308 for controlling the device. Software code is typically stored in memory 310 for execution by the processor 308 to control the various components of the device. The controller 306 and/or the pulse generator 304 may be connected to the electrodes 108 via leads or wirelessly.

The controller 306 also includes a user interface with input/output circuitry 312 adapted to allow a clinician to provide selection inputs or parameters to or more stimulation programs to treat the patient's disorder(s). Each stimulation program may include one or more sets of stimulation parameters including pulse amplitude, pulse width, pulse frequency, etc. The pulse generator 14 modifies its internal parameters in response to the control signals from controller device 16 to vary the stimulation characteristics of stimulation pulses transmitted through lead 303 to the patient. Any suitable type of pulse generating circuitry may be employed including constant current, constant voltage, multiple-independent current or voltage sources, etc. The energy applied is a function of the current amplitude and pulse width. The system 300 may also include a recording device operable to receive signals from a recording electrode positioned according to the techniques provided herein.

The system 300 may be used for neurostimulation or recording as well as determining whether an electrode has been properly positioned. For example, when the pulse generator 304 applies energy pulses, the effect of the stimulation is assessed to determine if the electrode is in proper contact with the nerve at step. For example, the signal at a recording electrode may be assessed and compared to empirical or estimated data to determine of the stimulation has occurred. If the electrode is in position, the biocompatible adhesive 104 and the biocompatible insulator 110 are applied to anchor the electrode in place. If the stimulation is not successful and the electrode is not well-positioned, the electrode may be moved to a second position on the nerve. For example, a first position on the nerve may not be successful because the electrode is not close enough to the nerve or because the nerve is damaged. A second location or position may improve the stimulation effects. The second position may be different than the first position, The controller may store the operating parameters for the calibration in a memory for execution during a calibration or a positioning mode.

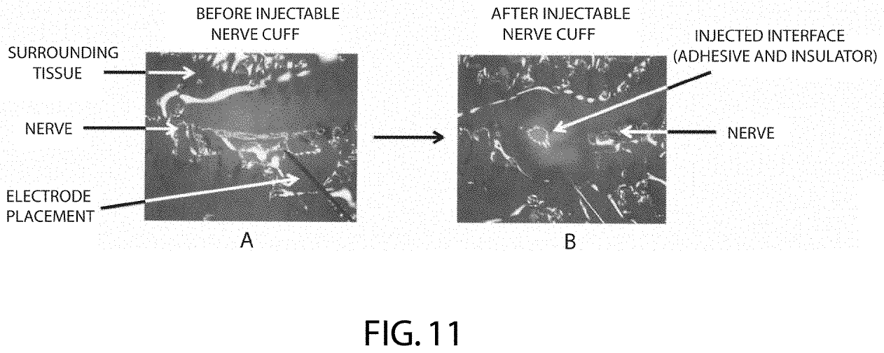

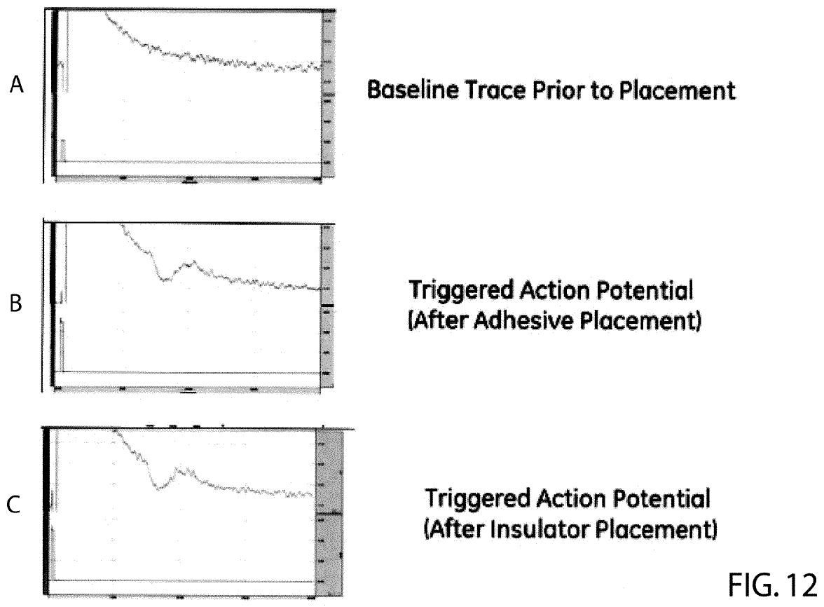

FIG. 11A shows an image of a needle used to place an electrode before injection of the adhesive and insulator components and FIG. 11B is an image after injection of the adhesive and insulator components. The silicone material used for insulation is shown to fully encapsulate the electrode after injection. FIGS. 12A-C show a neural recording prior to stimulation (FIG. 12A), after adhesive injection and electrical stimulation action potential response (FIG. 12B) and after insulator injection action potential response shown (FIG. 12C).

### Neural Stimulation

The human nervous system is a complex network of nerve cells, or neurons, found centrally in the brain and spinal cord and peripherally in the various nerves of the body. Neurons have a cell body, dendrites and an axon. A nerve is a group of neurons that serve a particular part of the body. Nerves may contain several hundred neurons to several hundred thousand neurons. Nerves often contain both afferent and efferent neurons. Afferent neurons carry signals back to the central nervous system and efferent neurons carry signals to the periphery. A group of neuronal cell bodies in one location is known as a ganglion. Electrical signals are conducted via neurons and nerves. Neurons release neurotransmitters at synapses (connections) with other nerves to allow continuation and modulation of the electrical signal. In the periphery, synaptic transmission often occurs at ganglia.

The electrical signal of a neuron is known as an action potential. Action potentials are initiated when a voltage potential across the cell membrane exceeds a certain threshold. This action potential is then propagated down the length of the neuron. The action potential of a nerve is complex and represents the sum of action potentials of the individual neurons in it.

Various stimulation patterns, ranging from continuous to intermittent, can be utilized. With intermittent stimulation, energy is delivered for a period of time at a certain frequency during the signal-on time. The signal-on time is followed by a period of time with no energy delivery, referred to as signal-off time.

Superimposed on the stimulation pattern are the treatment parameters, frequency and duration. The treatment frequency may be continuous or delivered at various time periods within the day or week. The treatment duration may last for as little as a few minutes to as long as several hours. Treatment duration with a specified stimulation pattern may last for one hour. The stimulation pattern may comprise various combinations of pulse width (the duration of a single pulse) and frequency (the interval between neighboring pulses). The treatment duration and frequency can be tailored to achieve the desired result.

Pulse generation for electrical nerve modulation is accomplished using a pulse generator. Pulse generators can use conventional microprocessors and other standard electrical components. A pulse generator for this embodiment can generate an energy pulse, or an energy signal, at frequencies ranging from approximately 0.5 Hz to 300 Hz, a pulse width from approximately 10 to 1,000 microseconds, and a constant current with amplitude of between approximately 0.1 milliamperes to 20 milliamperes. The pulse generator may be capable of producing a ramped, or sloped, rise in the current amplitude. The pulse generator can communicate with an external programmer and or monitor.

Bipolar stimulation of a nerve can be accomplished with multiple electrode assemblies with one electrode serving as the positive node and the other serving as a negative node. In this manner nerve activation can be directed primarily in one direction (unilateral), such as efferently, or away from the central nervous system. Unipolar stimulation can also be performed. As used herein, unipolar stimulation means using only a single electrode on the lead (a lead electrode), while an implanted pulse generator itself, or a ground electrode essentially functions as a second electrode, remote from the first electrode (a remote electrode). With unipolar stimulation, a larger energy field is created in order to electrically couple the electrode on the lead with the remote electrode. This allows successful nerve stimulation with the single electrode placed only in “general proximity” to the nerve, meaning that there can be significantly greater separation between the electrode and the nerve than the “close proximity” required for bipolar stimulation. The magnitude of the allowable separation between the electrode and the nerve will necessarily depend upon the actual magnitude of the energy field which the operator generates with the lead electrode in order to couple with the remote electrode. Accordingly, the present techniques may permit stimulation in areas where previously only unipolar, non-adjacent electrodes were used.

Provided herein are techniques to position electrodes on nerves in the context of a bi-directional neural interface. In one embodiment, the electrodes may facilitate modulation of neural pathways to produce a therapeutic outcome. Modulation may take place through direct electrical stimulation of the nerve/neural pathway (i.e. an implanted stimulation device with electrode)

Technical effects of the present disclosure include improved placement of electrodes for neural stimulation. For example, the disclosed techniques permit placement of electrodes in direct contact with the nerve for smaller peripheral nerves without using more damaging suturing techniques or without using neural cuffs that are more difficult to place.

The specific embodiments described above have been shown by way of example, and it should be understood that these embodiments may be susceptible to various modifications and alternative forms. It should be further understood that the claims are not intended to be limited to the particular forms disclosed, but rather to cover all modifications, equivalents, and alternatives falling within the spirit and scope of this disclosure.

1. A method of assembling a neural interface, comprising:
positioning an electrode proximate to a nerve such that the electrode is positioned partially proximate to or about an outer circumference of the nerve;
applying an injectable biocompatible adhesive between the electrode and the nerve; and
applying an injectable biocompatible insulator around the biocompatible adhesive, the nerve, and the electrode to provide in situ assembly of a neural interface around the nerve.

2. The method of claim 1, wherein the biocompatible adhesive is applied before the positioning of the electrode.

3. The method of claim 1, wherein the biocompatible adhesive is applied after the positioning of the electrode.

4. The method of claim 1, wherein applying the biocompatible adhesive comprises forming a gap between the nerve and the electrode that is at least partially filled by the biocompatible adhesive.

5. The method of claim 1, further comprising: positioning a second electrode proximate to the nerve and spaced apart from the first electrode and wherein applying the biocompatible adhesive comprises applying the biocompatible adhesive between the nerve and the second electrode.

6. The method of claim 5, wherein applying the biocompatible insulator around the biocompatible adhesive, the nerve, and the electrode comprises applying the biocompatible insulator around the second electrode.

7. The method of claim 1, wherein one or both of injectable biocompatible insulator or the injectable biocompatible insulator is applied via an injection device.

8. The method of claim 1, wherein the biocompatible adhesive is one of a gel, a liquid, or a foam.

9. The method of claim 1, wherein the biocompatible adhesive is one or more of fibrin, chitosan, polyvinylpyrrolidone, pyroxylin/nitrocellulose or poly(methylacrylate-isobutene-monoisopropylmaleate), or an acrylate or siloxane polymer.

10. The method of claim 1, wherein the biocompatible insulator comprises a silicone or cyanoacrylate polymer.

11. The method of claim 1, comprising activating the biocompatible insulator with a polymerization agent to polymerize around the biocompatible adhesive.

12. The method of claim 1, comprising allowing the biocompatible insulator to cure after the applying before applying an energy pulse to the electrode.

13. The method of claim 1, comprising causing an electrical signal to be applied to the electrode to stimulate the nerve before applying the biocompatible insulator to determine a quality of adhesion.

14. The method of claim 13, comprising repositioning the electrode when the quality of adhesion does not result in a received signal from the electrode or from a second electrode spaced apart from the electrode.

15. The method of claim 1, wherein the neural interface is a nerve cuff.

16. The method of claim 1, wherein the electrode forms a partial annulus about the nerve.

17. The method of claim 1, wherein the biocompatible adhesive is more porous than the biocompatible insulator.

18. The method of claim 1, wherein the biocompatible adhesive is more water absorptive than the biocompatible insulator.

19. The method of claim 1, wherein the biocompatible insulator is harder than the biocompatible adhesive in after the nerve cuff is assembled and the biocompatible insulator has cured.

20. A method of assembling a neural interface comprising:
positioning an implantable electrode device comprising an electrode proximate to a nerve such that the electrode is positioned partially proximate to or about an outer circumference of the nerve and wherein the implantable electrode device comprises a biocompatible adhesive layer on a surface of the electrode such that the biocompatible adhesive layer is positioned between the electrode and the nerve after the positioning; and
applying an injectable biocompatible insulator around the biocompatible adhesive layer, the nerve, and the electrode to provide in situ assembly of a neural interface around the nerve.

21. A kit for positioning an electrode on a nerve, the kit comprising:
a first applicator chamber holding a biocompatible adhesive and comprising a first tip through which the biocompatible adhesive is applied;
a second applicator chamber holding a biocompatible insulator or a precursor material that forms a biocompatible insulator and comprising a second tip through which the biocompatible insulator or the precursor material is applied; and
an implantable electrode device positioned proximate to the first tip and the second tip.

22. The kit of claim 21, comprising an activating agent for activating the precursor material to form the biocompatible insulator.

23. The kit of claim 21, wherein the first applicator chamber and the second application chamber are within a single injection device.

24. The kit of claim 21, wherein the first applicator chamber is part of a first injection device and the second application chamber is part of a second injection device separate from the first injection device.

25. The kit of claim 21, wherein the implantable electrode device comprises a stimulating electrode, a recording electrode, and a ground electrode.

26. A system for providing a bi-directional neural interface, comprising:
an electrode configured to be positioned on a nerve;
a substrate holding the electrode;
a biocompatible insulator configured to electrically isolate the electrode when the electrode is positioned on the nerve, wherein the biocompatible insulator is cured in place about the electrode; and
one or more of a pulse generator configured to deliver energy pulses to the electrode or a recording device.

27. The system of claim 26, further comprising a biocompatible adhesive configured to adhere the electrode to the nerve.

28. The system of claim 26, wherein the pulse generator comprises a controller storing a plurality of operating modes in a memory and that are executed by a processor.

25. The system of claim 24, comprising a recording electrode spaced apart from the electrode, and wherein one of the operating modes is a calibration mode configured to determine if the recording electrode is positioned to detect stimulation of the nerve via the electrode.

26. The system of claim 22, wherein the electrode is within an implantable electrode device comprising a plurality of electrodes spaced apart from one another.

27. The system of claim 22, wherein the electrode is disk-shaped.

28. The system of claim 22, wherein at least a portion of the electrode is curved or bent, and wherein the curved or bent portion of the electrode is positioned on the nerve.

29. An injection device for positioning an electrode on a nerve, comprising:
a first channel coupled to a reservoir holding a biocompatible adhesive and comprising a first tip through which the biocompatible adhesive is applied;
a second channel coupled to a reservoir holding a biocompatible insulator or a precursor material that forms a biocompatible insulator and comprising a second tip through which the biocompatible insulator or the precursor material is applied; and
an implantable electrode device positioned proximate to the first tip and the second tip.
